(19) United States
(12) Patent Application Publication
(10) Pub. No.: US 2014/0031684 A1
(43) Pub. Date: Jan. 30, 2014

(54) SYSTEM FOR TRANSCRANIAL ULTRASOUND IMAGING

(71) Applicant: Lidror TROYANSKY, Givataim (IL)

(72) Inventor: Lidror TROYANSKY, Givataim (IL)

(21) Appl. No.: 13/954,013

(22) Filed: Jul. 30, 2013

Related U.S. Application Data
(60) Provisional application No. 61/677,335, filed on Jul. 30, 2012.

(51) Int. Cl. A61B 8/08; A61B 8/00

(52) U.S. Cl. CPC A61B 8/0808; A61B 8/461
USPC 600/437

(57) ABSTRACT

Apparatus for transcranial imaging comprises an ultrasound source, placed in conjunction with a cranium, producing ultrasound at one or more ultrasound wavelengths that are focused on a location of interest within the cranium. The ultrasound generates RF radiation using the acousto-electric effect and a radio receiver detects the resulting radio frequency radiation emanating from the location of interest. Different types of brain tissue, as well as healthy and diseased tissue, produce different amplitudes of RF radiation, which can be detected as the location of interest is scanned over the volume of the brain, and used to produce an image of the brain.

### RELATED APPLICATION

This application claims the benefit of priority under 35 USC §119(e) of U.S. Provisional Patent Application No. 61/677,335 filed Jul. 30, 2012, the contents of which are incorporated herein by reference in their entirety.

### FIELD OF INVENTION

The invention relates generally to systems for imaging the human brain, and more particularly but not exclusively to a way of using ultrasound in such imaging.

### BACKGROUND

High quality, non-invasive imaging of the human brain is of tremendous importance for the diagnosis of brain tumors, lesions and pathologies and for improved understanding of the different functions and faculties of the human brain.

Current imaging methods, such as Magnetic Resonance Imaging (MRI), Computerized Tomography and Positron Emission Tomography are very expensive, cumbersome or require special preparation.

Attempts to use ultrasound for imaging of adult brain have gained, so far, very limited success, due, inter alia, to the following reasons:

The human brain is shielded by the massive bones of the cranium (skull) and the brain meninges, which greatly reduces the amount of the transmitted acoustical energy that penetrates into the brain tissues and attenuates the received acoustic signal, therefore significantly reducing the signal to noise ratio.

The acoustic energy is exponentially attenuated while traversing to and from the target tissue, which, again, reduces the signal-to-noise ratio.

The contrast mechanism is based on the subtle differences between the refractive indices of soft tissues in the brain, which imply that only a small fraction of the energy is reflected from the target tissues.

The skull bones hamper the ability to focus the acoustic pulses, which reduces the achievable resolution.

The reflected echoes convey very little information regarding the functional level of the target tissues.

Reverberations from the tissues farther complicate the interpretation of the reflected echoes.

Ultrasound imaging of the brain is currently used for fetuses and for babies shortly after birth, where openings in the skull are present, but cannot be used once the skull is fully formed.

Nevertheless, ultrasound has particular advantages in imaging soft tissue. This includes being safe at the energy levels needed, requiring only relatively inexpensive equipment and not requiring isolation of the patient within a large and forbidding apparatus such as with MRI.

There is therefore a clear need, and it would be highly valuable to have, a system and a method for imagining a human brain, that would overcome the limitations of the current methods.

### SUMMARY OF THE INVENTION

The present invention relates to use of ultrasound for transcranial imaging, that is to say imaging of the brain through a fully-formed skull. The ultrasound is focused at the location of interest within the brain and detection is of resulting RF radiation which may be caused by the so-called electro-acoustic effect, as charged particles, ions within and between the nerve tissue, are caused to vibrate by the ultrasound. Embodiments thus provide a bimodal brain imaging system.

In the electro-acoustic effect, ultrasound waves induce oscillations of the neurons in the target tissues. Neurons typically comprise a surplus of positive potassium ions, which are relatively small and light, that are balanced by significantly larger and heavier organic anions. Under the influence of the ultrasound wave the positive, potassium, ions and the negative, organic, ions have different displacement amplitudes along the longitudinal wave of the ultrasound. This difference, as well as the induced oscillation of the neuronal membranes and other electroacoustic phenomena, create an alternating electric potential between various points in the ultrasound wave.

Consequentially, an electromagnetic wave is created having the same frequency as the ultrasound wave, thereby indicating the existence and location of intact neurons in the target tissue and allowing differentiation between different types of tissues. In particular, since the white matter in the brain is composed largely of isolated lipid tissues composed by the glial cells, with lower mobility and smaller concentration of ions, the amplitude of the induced oscillations is smaller than with gray matter, and the overall amplitude of the RF is thus smaller. Thus the system allows differentiation between the white matter and gray matter.

The system may detect the induced radio frequency (RF) using a narrowband receiver tuned to the frequency of the ultrasound waves, thereby improving the signal-to-noise ratio.

The system may sample the amplitude and the power of the RF signal with a high temporal resolution, in order to achieve high spatial resolution.

In accordance with some embodiments the system may utilize two scanning ultrasound beams with different frequencies, which, due to nonlinearities in the tissues, produce oscillations with frequencies that are equal to the sum or the difference of the frequencies of the two beams. The system uses the electromagnetic signals of these frequencies in order to determine the structure and the level of activity in the intersection area of the two beams.

In some embodiments three beams with different frequencies may be used in order to allow improved spatial resolution.

In accordance with some embodiments the system may comprise a module that measures the power of the electromagnetic radiation in two orthogonal directions of polarization and compares the two orthogonal directions of polarization. This comparison allows direct inference of the density of intact neurons and their activity level in the target tissues.

In accordance with some embodiments the system may measure the received electromagnetic signal in a selected area or areas of the brain of a subject while the subject performs different cognitive or motor tasks and compares them with the signal level when the subject is at rest. It is thus possible to detect levels of activity in the selected area or areas, that were induced by the task.

According to an aspect of the present invention there is provided apparatus for transcranial imaging, comprising:

an ultrasound source, for location in conjunction with a cranium and configured to produce ultrasound at one or more predetermined ultrasound frequencies and focusable on a location of interest within the cranium; and

a radio receiver configured to detect radio frequency radiation emanating from the location of interest, thereby to allow detection of RF radiation induced at the location of interest by the ultrasound source, for insertion into an image.

In an embodiment, the radio receiver is tuned to a waveband that includes the one or more predetermined ultrasound frequencies, or, where there is more than one frequency in use, their sum and difference frequencies as well.

In an embodiment, the radio receiver is tuned to the one or more predetermined ultrasound frequencies, and sum and difference frequencies where relevant.

In an embodiment, the ultrasound source is configured to produce two or more beams, each at a different respective ultrasound frequency, and each focused on the location of interest, to form sum and difference frequencies at the location of interest.

In an embodiment, the ultrasound source is configured to produce three beams, each at a respectively different ultrasound frequency, and each focused on the location of interest, to form sum and difference frequencies at the location of interest.

In an embodiment, one of the beams is orthogonal to one of the other beams.

In an embodiment, the radio receiver is connected to an analysis unit, the analysis unit configured to identify one member of the group consisting of amplitudes, power, phase, a combination of amplitude and power, a combination of amplitude and phase, a combination of power and phase, and a combination of amplitude, power and phase, within the radio signal and process the member into images of brain tissue.

In an embodiment, the radio receiver comprises a plurality of antennas with respectively different polarizations and/or a plurality of antennas positioned to detect the RF signal with different attenuations. The signal is attenuated but the noise may stay the same between the antennas, allowing for noise cancellation.

An embodiment may comprise an acoustic barrier located at a distance from the cranium, the cranium being a whole number of wavelengths of the predetermined frequency.

The embodiments further relate to a transcranial image created using the presently disclosed apparatus.

According to a second aspect of the present embodiments there is provided a method of transcranial imaging, and an image created using the method, the method comprising:

targeting ultrasound of one or more predetermined frequencies at a location of interest within a cranium; and

detecting radio waves emanating from within the cranium during the targeting.

According to a third aspect of the present embodiments there is provided a system for transcranial imaging of a target tissue in a brain comprising:

a control module for producing a signal at a predetermined frequency in an ultrasound frequency range;

a transducer for transducing the signal to an ultrasound signal;

a focusing mechanism for focusing the ultrasound signal onto a target within the transcranial region; and

a radio frequency receiver for detecting an electromagnetic signal at a frequency band that includes the predetermined frequency.

In accordance with some embodiments the system utilizes the non-linear response of the brain tissues and fluids in order to create parametric focusing of the beam, thereby maintaining the unique resolution of high frequencies, while obtaining lower attenuation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a simplified block diagram illustrating apparatus according to a first embodiment of the present invention;

FIG. 1B is a simplified block diagram of a system for transcranial imaging of a human brain in accordance with an embodiment of the invention;

FIG. 2 is a simplified schematic illustration of the system of FIG. 1 illustrating a mechanism for induction of electromagnetic waves in neuronal tissue by ultrasound waves in accordance with an embodiment of the invention;

FIG. 3 is a simplified schematic illustration of the system of FIG. 1 illustrating an RF receiver of the system in accordance with an embodiment of the invention;

FIG. 4 is a simplified schematic illustration of the system of FIG. 1 illustrating an RF receiver of the system in accordance with another embodiment of the invention;

FIG. 5 is a simplified schematic illustration of the system of FIG. 1 utilizing two transmitters emitting directed ultrasound beams, each with a different frequency and utilizing nonlinear effects for improved spatial resolution in accordance with an embodiment of the invention;

FIG. 6 is a simplified schematic illustration of the system of FIG. 1 utilizing two transmitters emitting directed ultrasound pulses for improved spatial resolution in accordance with an embodiment of the invention;

FIG. 7 is a simplified schematic illustration of the system of FIG. 1 illustrating three beams with different frequencies used for improved spatial resolution in accordance with an embodiment of the invention; and

FIG. 8 is a simplified schematic illustration of the system of FIG. 1 utilizing the principle of “resonant tunneling” (or the optical Fabry-Perot resonator) for improved penetration of the ultrasound waves through the skull bones in accordance with an embodiment of the invention.

### DETAILED DESCRIPTION OF CERTAIN EMBODIMENTS

The present embodiments provide an apparatus for transcranial imaging, which comprises an ultrasound source placed in conjunction with a cranium, producing ultrasound at one or more ultrasound wavelengths that are focused on a location of interest within the cranium. The ultrasound generates RF radiation using an acousto-electric effect, and a radio receiver detects the resulting radio frequency radiation emanating from the location of interest. Different types of brain tissue, as well as healthy and diseased tissue, produce different amplitudes or powers or phases of RF radiation, which can be detected from different locations during an ultrasound scan and used to produce an image of the brain. Electroacoustic phenomena arise when ultrasound propagates through a fluid containing ions. The associated particle motion generates electric signals because ions have electric charge. This coupling between ultrasound and electric field is referred to under the general heading of electroacoustic phenomena. Fluid might be a simple Newtonian liquid, or a complex heterogeneous dispersion, an emulsion or even a porous body. There are several different electroacoustic effects depending on the nature of the fluid.

Historically, the ion vibration current (IVI) is the first known electroacoustic effect. It was predicted by Debye in 1933. He pointed out that the difference in the effective mass or friction coefficient between an anion and a cation would result in different displacement amplitudes in a longitudinal wave. This difference creates an alternating electric potential between various points in sound wave. This effect was extensively used in 1950's and 1960s for characterizing ion solvation. These works are mostly associated with the names of Zana and Yaeger, who published a review of their studies in 1982.

Brain tissues are not conventionally considered as a fluid, although they are full of anions and cations, and thus the significance of the effect to brain tissues has apparently not heretofore been appreciated, certainly not in connection with brain imaging.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In this description, reference is made to the drawings wherein like parts are designated with like numerals throughout.

The following embodiments describe systems, methods, and apparatus for effective transcranial imaging of the brain (human or animal), particularly but not exclusively, in a bi-modal manner, where ultrasound waves induce oscillations of charged particles inside and outside the cells in target tissues. Such oscillations, in turn, produce electromagnetic radiation with frequencies that correspond to the frequencies of the ultrasound waves, in accordance with the acousto-electric effect described above. The amplitude and the power of radiation depends on the density and the types of charged particles in the target tissues, thereby conveying significant information pertaining to the composition and the state of the target tissues.

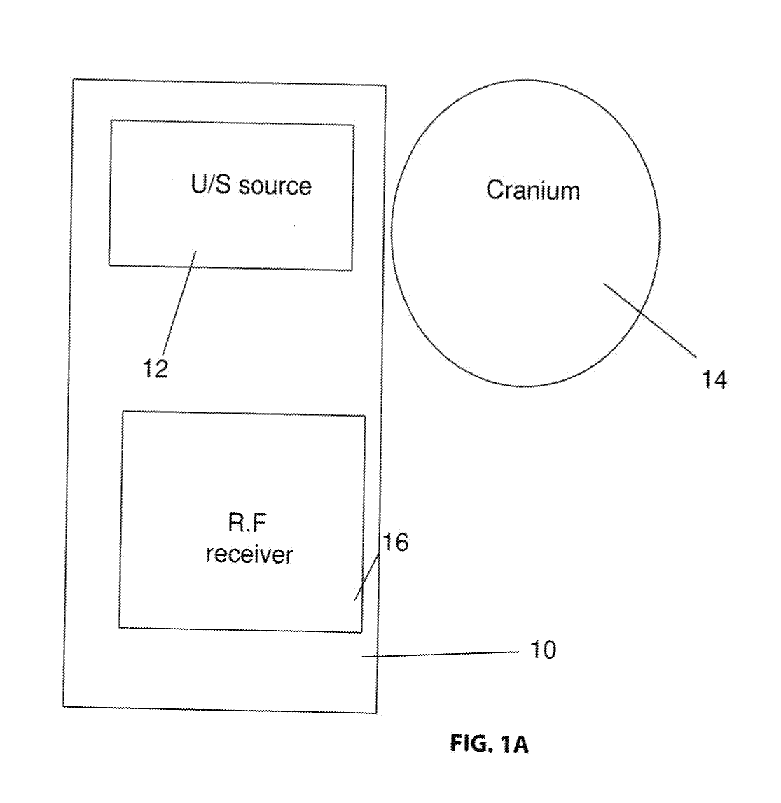

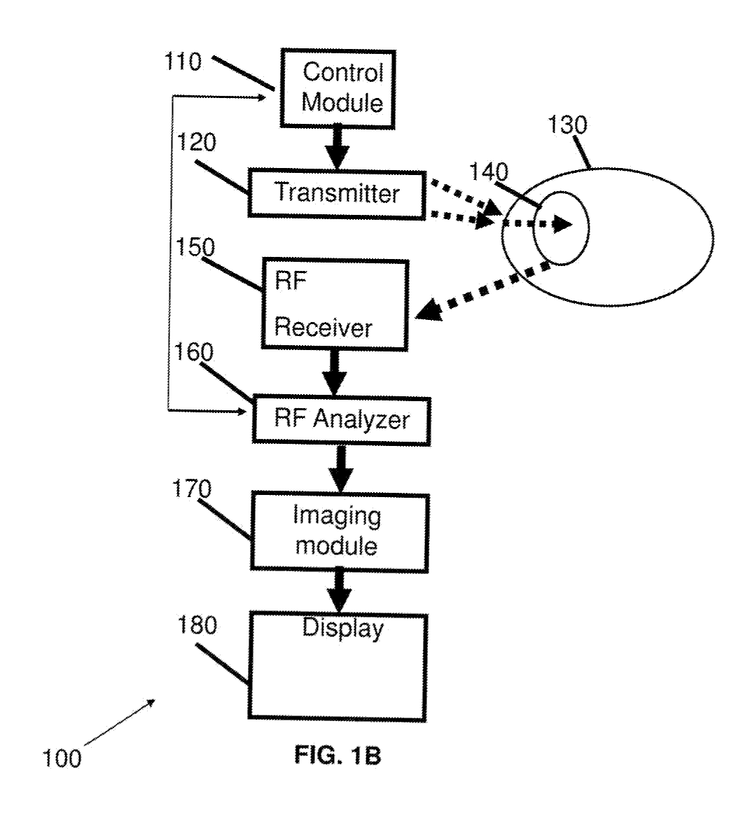

Reference is now made to FIG. 1A, which illustrates apparatus 10 for transcranial imaging. The apparatus includes an ultrasound source 12, which is placed in conjunction with cranium 14 and which produces ultrasound as a beam at a preset ultrasound frequency. The ultrasound is focused on a location of interest within the cranium. The ultrasound source may produce two or more beams at different frequencies and both focused on the same point of interest, so that sum or difference frequencies may operate at the focal point to add second order effects to the overall detection. Furthermore, effects of the beam away from the focus can more easily be ignored since substantially no sum or difference signals are experienced there. The points of interest may scan over some or all of the transcranial area over the course of a period of time, to provide partial or complete brain scans. The amplitude and/or power detected while any given location is the focus of the scan makes up that given location in the overall image. The overall image may be a two-dimensional or three-dimensional scan as with conventional ultrasound and with other imaging modes. As discussed, vibration of the ions in the brain tissue induces RF radiation which can be detected, and the different tissues have varying proportions of the different ions, giving rise to different amplitudes or power levels or phases or any combination of amplitudes, power and phase, of the RF radiation produced. In particularly, healthy and unhealthy tissues can be distinguished in this way.

A radio receiver 16 is used to detect radio frequency radiation emanating from the location of interest. The radio receiver may be a standard radio receiver tuned to the waveband of the ultrasound.

In an embodiment the radio receiver may be a directional receiver directed to the location of interest, thus reducing noise relating to background radiation coming from other directions.

The ultrasound source conventionally comprises a transducer and a beam former. The sound is focused either by the shape of the transducer, a lens in front of the transducer, or a complex set of control pulses from the ultrasound scanner machine (Beamforming). Focusing produces an arc-shaped sound wave from the face of the transducer. The wave travels into the body and comes into focus at a desired depth.

Older technology transducers focus their beam with physical lenses. Newer technology transducers use phased array techniques to enable the sonographic machine to change the direction and depth of focus. Almost all piezoelectric transducers are made of ceramic.

Materials on the face of the transducer enable the sound to be transmitted efficiently into the body. There is often provided a rubbery coating as a form of impedance matching. In addition, a water-based gel is placed between the patient's skin and the probe.

As discussed below, the radio receiver may be located within a Faraday cage to further suppress background radiation.

The radio receiver may be connected to an imaging processor or imaging unit, the processing unit may identify different amplitudes and/or powers and/or phases within the radio signal and process the results obtained at different target locations into images of brain tissue.

Turning now to FIG. 1B, a system 100 for transcranial imaging of the human brain is illustrated. A control module 110, which typically comprises a computing device, produces a signal with a typical frequency between substantially 500 KHZ and substantially 100 MHZ. The signal is transduced to an acoustic ultrasound signal by a transducer which is part of transmitter 120. The transmitter 120 may comprise multiple transducers and a beamformer as discussed above. The transmitter 120 sends a directional ultrasound beam to skull 130.

Some of the ultrasound energy transmitted to a target tissue 140 in the brain, causes the charged particles inside and outside the cells of the target tissue 140, such as neurons and ions in the intracellular fluid, to oscillate. The charged particle oscillation urges emission of electromagnetic waves with a frequency that is substantially equal to the frequency of the acoustic ultrasound signal. The electromagnetic energy is detected by an RF receiver 150. The amplitude and the power of the received RF signal as a function of time is analyzed by an RF analyzer module 160, which is typically in communication with the control module 110. Based on the analysis and the location information from the control module 110, an imaging module 170 can infer information pertaining to neuronal tissue mapping and create an image. The image may be displayed on a display 180. Such information may include, for example, whether the target tissue 140 comprises neurons or white matter, and whether the tissue contains lesions, tumors and the existence of regions with dead neurons, which no longer sustain membrane polarization.

In accordance with an embodiment, the amplitude of the received electromagnetic signal is calibrated against an estimate of the anticipated power of the ultrasound wave in the target tissue 140, which is based on previously known estimated transmission coefficients and an average attenuation in the brain.

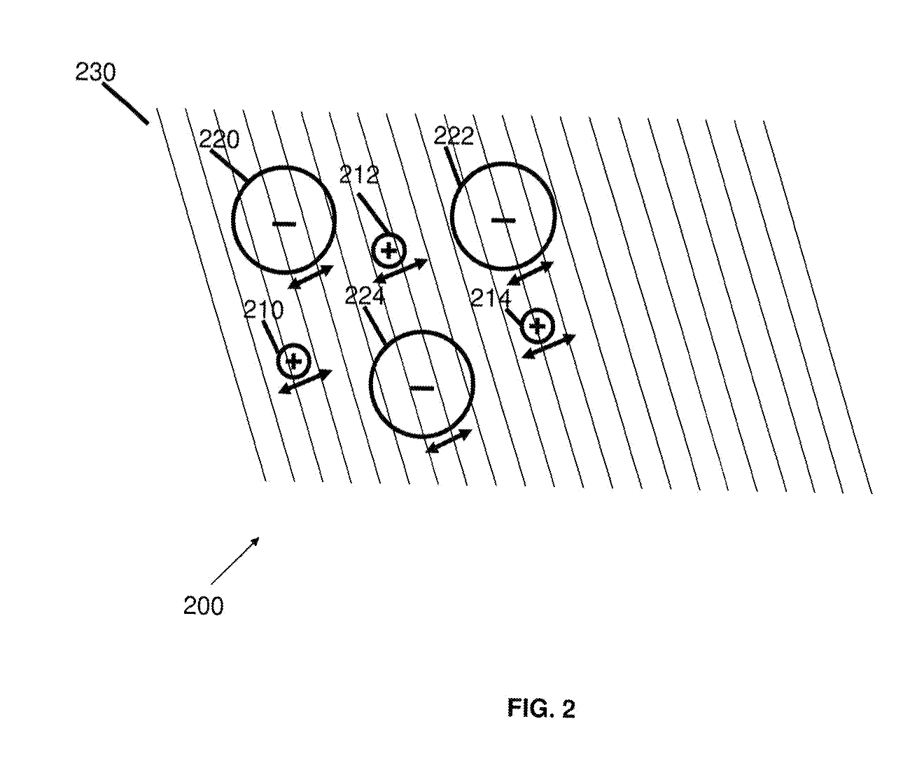

Turning now to FIG. 2, a mechanism for induction of electromagnetic waves in neuronal tissue by ultrasound waves is illustrated. An intracellular medium 200 contains Potassium ions 210, 212 and 214, and substantially larger and heavier organic anions 220, 222 and 224. Under the influence of an ultrasound wave 230, the positive ions 210, 212 and 214 and the negative ions 220, 222 and 224 have different displacement amplitudes along the longitudinal wave. This difference creates an alternating electric potential between various points in the ultrasound wave, which create an electromagnetic wave with the same frequency as the ultrasound wave.

As the polarization of the neurons requires the active mechanism of the sodium-potassium pump, regions in which a large portion of the neurons have died (e.g. due to a stroke) will produce, in general, less intensive electromagnetic waves. In addition, neuronal activities induce fluxes of sodium and potassium ions to and from the neurons, thereby altering the level of the electro-acoustic effect and the resulting amplitude of the emitted electromagnetic waves. An analysis of the received signal level during performance of various cognitive or motor tasks can thereby reveal the changes in the activity level in a region, which changes are induced by the cognitive or motor tasks.

In accordance with an embodiment, the system 100 detects the induced RF signal using a narrowband receiver tuned to the frequency of the ultrasound waves, thereby improving the signal-to-noise ratio. The system 100 can sample the amplitude and the power of the RF signal with a high temporal resolution, for achieving high spatial resolution.

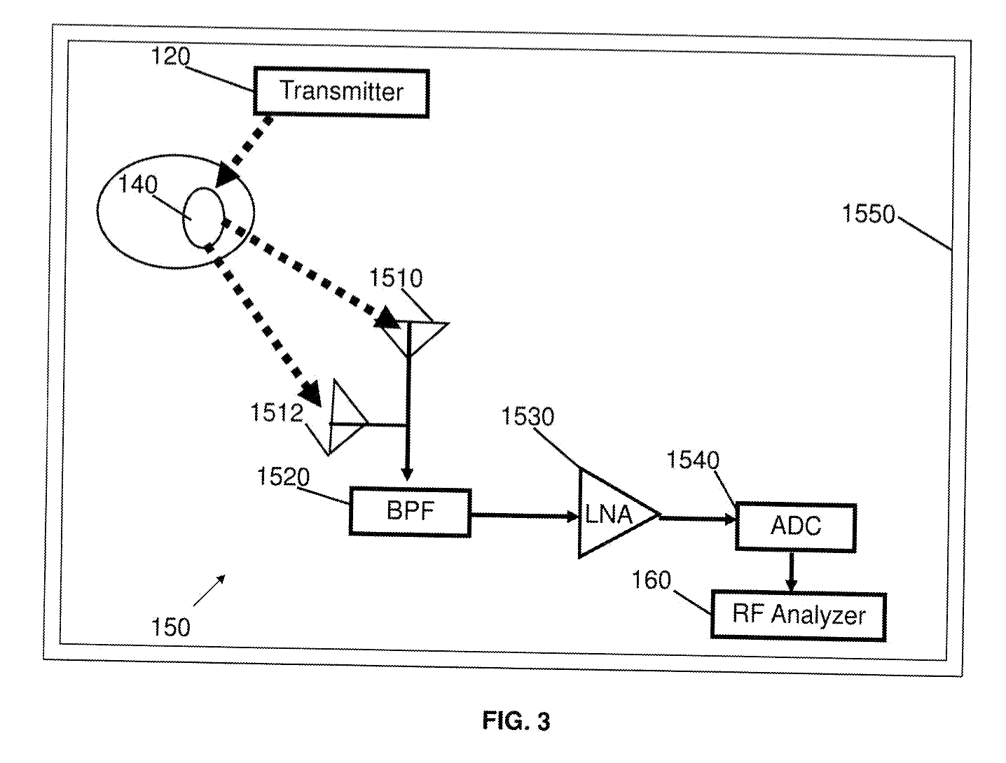

FIG. 3 illustrates the RF receiver 150 of FIG. 1B. The ultrasound transmitted by the transmitter 120 induces an alternating electric potential in the target tissue 140. The resultant electromagnetic waves are received by a first antenna 1510, and the signal is filtered by a bandpass filter 1520, which is tuned to transfer the frequencies induced by the ultrasound waves. The signal is amplified by a low-noise amplifier 1530 and is sampled, at a high sampling frequency, by an analog-to-digital component 1540. The digital signal is thereafter transferred to the RF analyzer 160 for further analysis.

To improve the signal-to-noise ratio the RF receiver 150 may be located inside a Faraday Cage 1550, as discussed above.

In accordance with an embodiment two or more antennas may be employed.

For example, a second antenna with horizontal polarization 1512 may be used along with a vertically polarized first antenna 1510. Use of multiple antennas allow identifying of different mechanisms that produce the electromagnetic radiation (e.g., motion of membranes vs. particle motion).

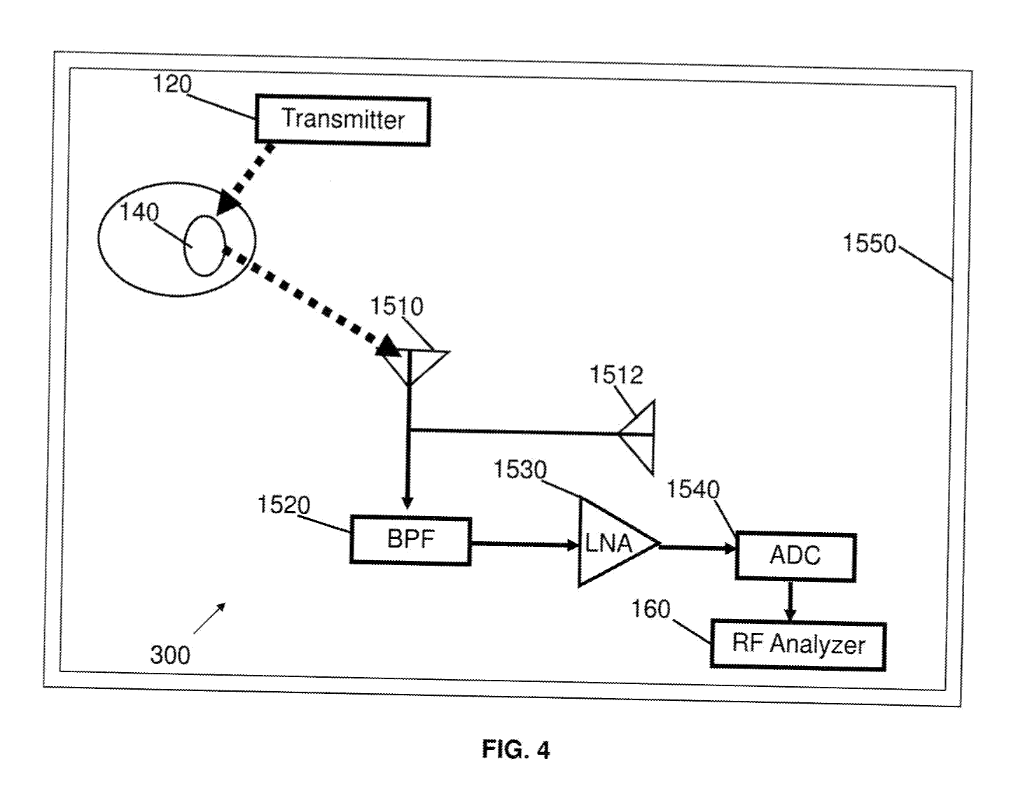

FIG. 4 illustrates an RF receiver 300, substantially similar to the RF receiver 150 of FIG. 3. The RF receiver 300 is a separate receiver for the second antenna 1512, and is positioned such that the signal received from the target tissue 140 is less strong than the signal received in the first antenna 1510, while the level of the background noise received in the first and second antennas 1510 and 1512, respectively, is substantially similar, thereby facilitating the usage of noise-cancelling algorithms.

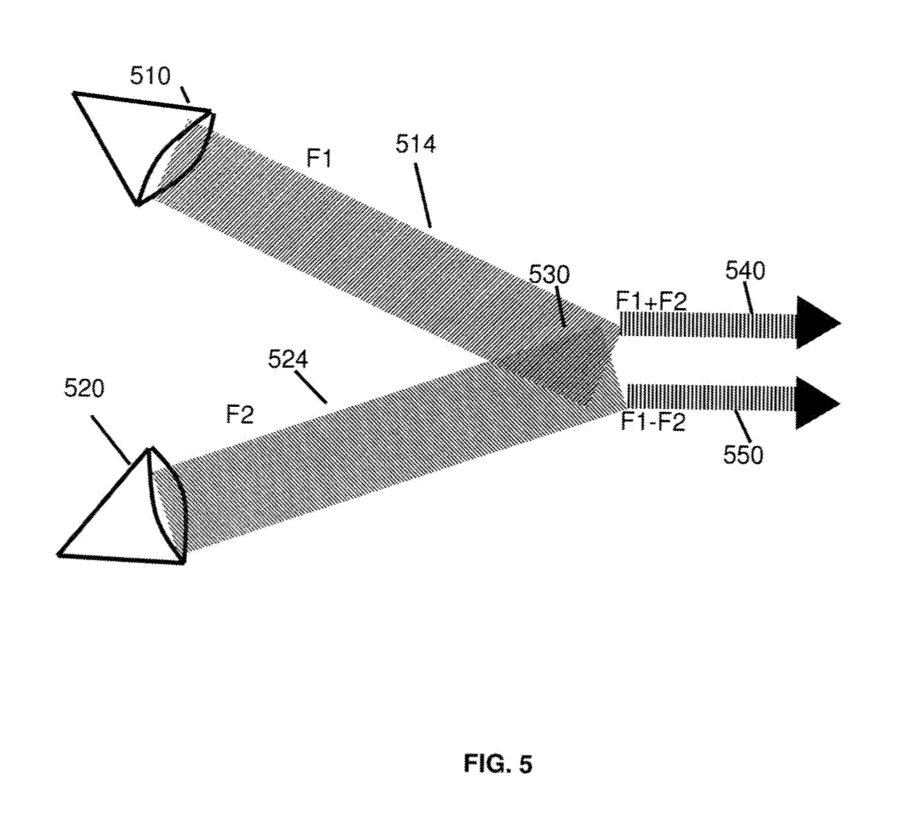

FIG. 5 is a simplified schematic illustration of the system 100 of FIG. 1B utilizing two transmitters emitting directed ultrasound beams. A first transmitter 510 emits an ultrasound beam 514 with a frequency F1, and a second transmitter 520 emits an ultrasound beam 524 with a different frequency F2. The two beams 514 and 524 intersect at a region 530. Due to nonlinear effects in the target tissue 140, the combined effect of the two beams 514 and 524 induces oscillations with frequencies of F1+F2 and |F1−F2|. These frequencies produce electromagnetic waves with corresponding frequencies 540 and 550, respectively. The power of the electromagnetic field may depend on the composition of the target tissues 140 in the intersection area, and, in particular, may depend on the nonlinearity of the medium at the intersection of the two beams 514 and 524 in region 530. Thereby, additional information regarding the structure and the health of the tissues in this region 530 is revealed. Furthermore, appropriate alignment of the two beams can ensure that the sum and difference frequencies occur only at the focal point, allowing for a simple way to discount noise effects of the beam at other locations in the brain.

In accordance with some embodiments the ultrasound waves in the two beams 514 and 524 may be continuous (CW), thereby allowing longer integration times and allowing signals with relatively narrower bandwidth, and thus significantly enhancing the signal-to-noise ratio.

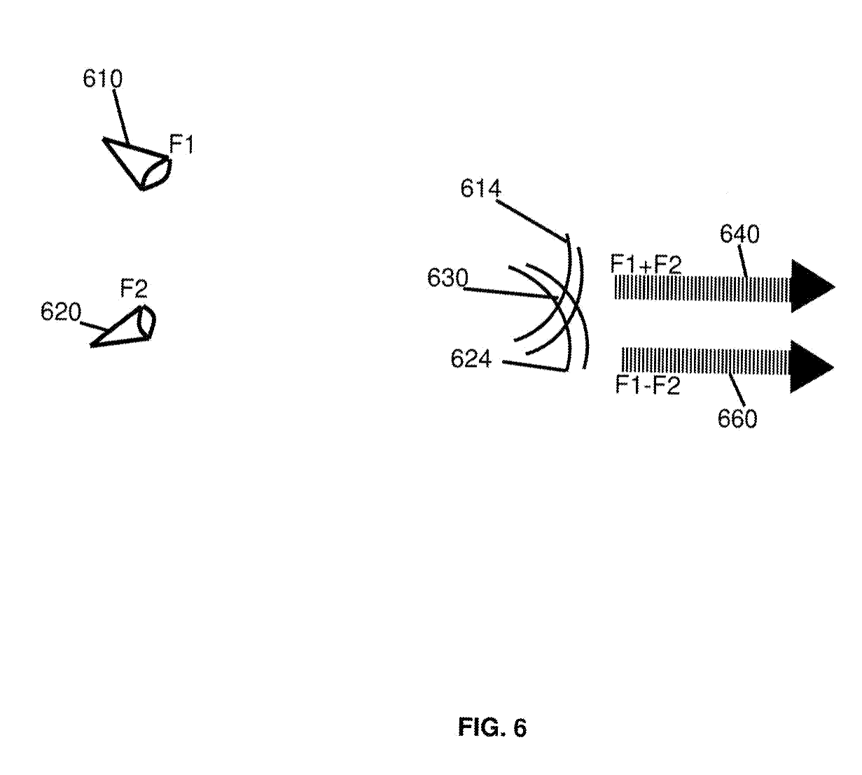

In accordance with some embodiments the two beams send pulses, and the intersection times are used in order to obtain improved location information regarding the target tissue 140, as illustrated in FIG. 6. FIG. 6 illustrates transmitters 610 and 620 which are substantially similar to the transmitters 510 and 520 in FIG. 5. The transmitter 610 emits a directional ultrasound pulse 614 with a frequency F1, and the transmitter 620 emits a directional ultrasound pulse 624 with a frequency F2. The two pulses intersect at a region 630, and, again, due to the nonlinear responses of the target tissues 140 in this region 630, oscillations with frequencies F1+F2 and |F1−F2| are induced and emit electromagnetic waves 640 and 660 with respective frequencies F1+F2 and |F1−F2|.

In accordance with some embodiments one of the beams may transmit continuously (CW), while the other beam may transmit short pulses. This combination of continuous and short pulse beam transmission may allow analysis of the patterns of the RF signals formed while the pulse traverses an area illuminated by the first beam. By using the timing information improved resolution may be obtained.

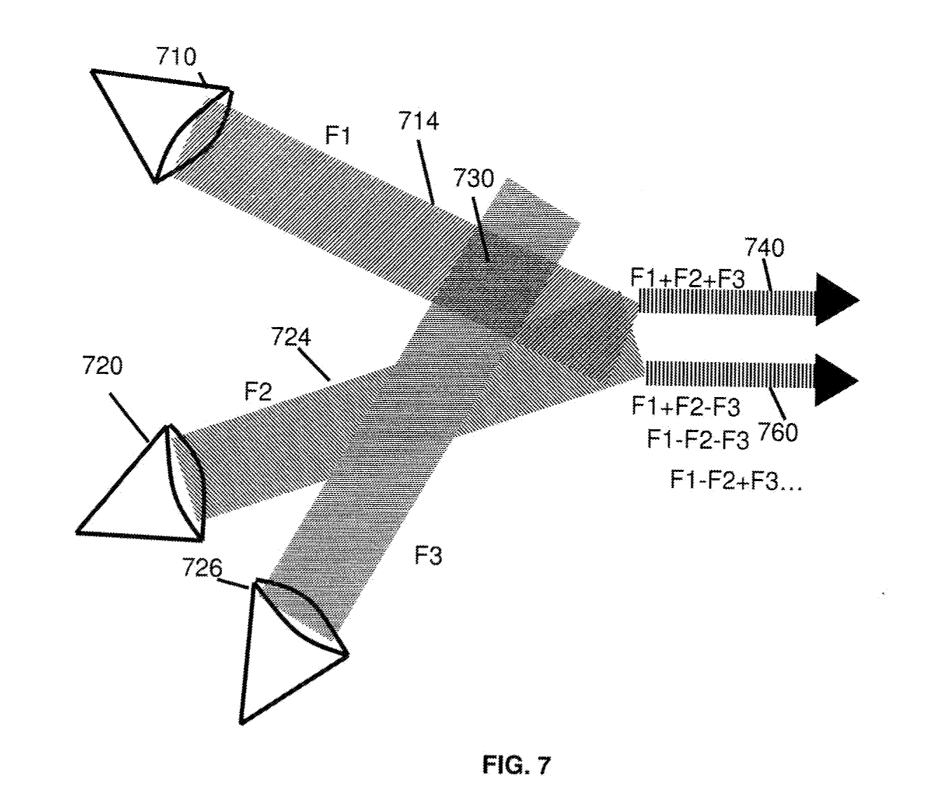

In accordance with some embodiments three beams with different frequencies are used in order to allow improved spatial resolution, as illustrated in FIG. 7A transmitter 710 emits a directional ultrasound beam 714 with a frequency F1, a transmitter 720 emits a directional ultrasound beam 724 with a frequency F2, and a transmitter 726 emits a directional ultrasound beam 728 with a frequency F3. The three beams 714, 724 and 728 intersect at a region 730. In this case, third-order effects may give rise to oscillations with the various combinations of the three frequencies abs(±F1±F2±F3) which mark the responses of the target tissues 140 in the intersection region 730 to the ultrasound waves. As seen in FIG. 7, a combination of the three frequencies F₁+F₂+F₃ designated by reference numeral 740 and a combination of the three frequencies F₁+F₂−F₃ designated by reference numeral 760, it being appreciated that many frequency combinations may be received. As the intersection region 730 of the three beams 714, 724 and 726 may be made arbitrarily small, a higher spatial resolution is therefore feasible.

In accordance with some embodiments the system 100 may utilize many different frequencies in order to scan various areas simultaneously, thereby significantly reducing the scanning time. In this case each transmitter may emit multiple signals with different frequencies, and the beamformer of each transmitter may simultaneously produce beams in a different direction for each of the frequencies, thereby allowing simultaneously scanning several regions.

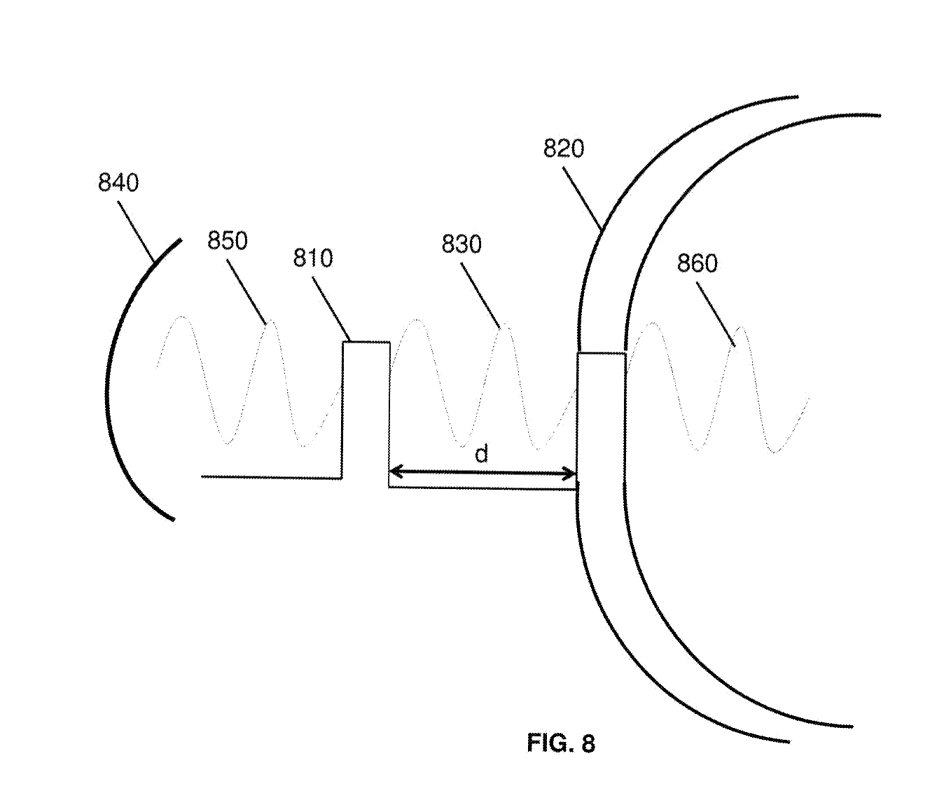

In accordance with some embodiments the system 100 may utilize a device that uses the principle of “resonant tunneling” (or the optical Fabry-Perot resonator) for enhanced penetration of the ultrasound waves through the skull bones, as illustrated in FIG. 8. As seen in FIG. 8, an acoustic barrier 810, which transfers a fraction T of the acoustic energy and reflects R=1−T of the acoustic energy, is located at a distance d from the skull 820. d is selected to be equal to an integer number of wavelength of the acoustic wave 830. The transmitter 840 sends ultrasound beam 850 towards the barrier. A resonance in the space between the barrier 810 and the skull 820 facilitates a higher penetration of the acoustic wave 860 behind the skull 820, thereby increasing the efficiency of the transmission.

In accordance with some embodiments the system 100 comprises a module that measures the power of the electromagnetic radiation in two orthogonal polarization directions and compares the two, thereby allowing direct inference of the motion of the charged particles in the direction of the acoustic ultrasound wave and in the perpendicular direction thereof.

In accordance with some embodiments the system 100 may measure the received electromagnetic signals in a selected area of the brain of the subject while the subject performs different cognitive or motor tasks and compares them with the signal level when the subject is at rest. It is thus possible to assess the level of activity in the selected area that was induced by the task.

In some embodiments the beamformer is used to focus the beam on the target tissue. In this case, the waves from the various transducers coherently converge on the target tissue, and the emitted RF waves provide the relevant information, as discussed above.

The system 100 described above may be used for a wide range of applications, including diagnosis of brain tumors, stroke, lesions and various other pathologies.

Moreover, the system 100 may be used to assist in brain surgeries and for gaining a better understanding of the organic basis of cognition and behavior. In addition, since the system 100 can sense activities in specific areas in the brain, it can also be used to operate external devices, such as a prosthesis.

It is appreciated that the system 100 described hereinabove may be used for imaging the human brain, an animal brain or any other relevant target tissue or medium of interest, and may be useful for scanning any ionized material concealed behind a casing. Indeed the acousto-electric effect works even better if there is no casing concealing the tissue.

The methods and apparatus disclosed herein provide methods and systems which may provide effective transcranial imaging. It is expected that during the life of a patent maturing from this application many relevant ultrasound transmitters and radio receivers will be developed and the scopes of the corresponding terms are intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment, and the above description is to be construed as if this combination were explicitly written. Conversely, various features of the invention, which are, for brevity, described in the context of a single embodiment, may also be provided separately or in any suitable subcombination or as suitable in any other described embodiment of the invention, and the above description is to be construed as if these separate embodiments were explicitly written. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. Apparatus for transcranial imaging, comprising:
an ultrasound source, for location in conjunction with a cranium and configured to produce ultrasound at one or more predetermined ultrasound frequencies and focusable on a location of interest within said cranium; and
a radio receiver configured to detect radio frequency radiation emanating from said location of interest, thereby to allow detection of RF radiation induced at said location of interest by said ultrasound source, for insertion into an image.

2. The apparatus of claim 1, wherein said radio receiver is tuned to a waveband that includes said one or more predetermined ultrasound frequencies.

3. The apparatus of claim 1, wherein said radio receiver is tuned to said one or more predetermined ultrasound frequencies.

4. The apparatus of claim 1, wherein said ultrasound source is configured to produce two or more beams, each at a different respective ultrasound frequency, and each focused on said location of interest, to form sum and difference frequencies at said location of interest.

5. The apparatus of claim 1, wherein said ultrasound source is configured to produce three beams, each at a respectively different ultrasound frequency, and each focused on said location of interest, to form sum and difference frequencies at said location of interest.

6. The apparatus of claim 4, wherein one of said two or more beams is orthogonal to at least one other of said two or more beams.

7. The apparatus of claim 1, wherein said radio receiver is connected to an analysis unit, the analysis unit configured to identify one member of the group consisting of amplitudes, power, phase, a combination of amplitude and power, a combination of amplitude and phase, a combination of power and phase, and a combination of amplitude, power and phase, within the radio signal and process said member into images of brain tissue.

8. The apparatus of claim 1, wherein said radio receiver comprises one member of the group consisting of a plurality of antennas with respectively different polarizations and a plurality of antennas positioned to detect said RF signal with different attenuations.

9. The apparatus of claim 1, further comprising an acoustic barrier located at a distance from said cranium, said cranium being a whole number of wavelengths of said predetermined frequency.

10. A transcranial image created using the apparatus of claim 1.

11. A method of transcranial imaging comprising:
targeting ultrasound of one or more predetermined frequencies at a location of interest within a cranium; and
detecting radio waves emanating from within said cranium during said targeting.

12. The method of claim 11, further comprising extracting at least one member of the group consisting of amplitude, power, phase, a combination of amplitude and power, a combination of amplitude and phase, a combination of power and phase, and a combination of amplitude, power and phase, from said detected radio waves and forming images based on said detected member.

13. A transcranial image created using the method of claim 11.

14. A system for transcranial imaging of a target tissue in a brain comprising:
a control module for producing a signal at a predetermined frequency in an ultrasound frequency range;
a transducer for transducing the signal to an ultrasound signal;
a focusing mechanism for focusing the ultrasound signal onto a target within the transcranial region; and
a radio frequency receiver for detecting an electromagnetic signal at a frequency band that includes said predetermined frequency.

15. The system of claim 14, further comprising a radio frequency analyzer module for analyzing the electromagnetic signal.

16. The system of claim 15, wherein said radio frequency analyzer module is configured to analyze said electromagnetic signal into one member of the group consisting of different amplitudes, different powers, different phases, a combination of different amplitudes and different powers, a combination of different powers and different phases, a combination of different amplitude and different phases, and a combination of different amplitudes, different powers and different phases.

17. The system of claim 16, further comprising an imaging module for receiving information based on said analysis of the electromagnetic signal and creating an image based on said member as it differs over a course of a scan.

18. The system of claim 15, comprising a display associated with said imaging module for displaying said image.

19. The system of claim 14, wherein a plurality of ultrasound signals, each at a respectively different frequency, is focused on the target.

20. The system of claim 14, wherein the ultrasound signal comprises two beams, each at a respectively different frequency, one being a continuous signal and one being a pulsed signal.
